(19) United States
(12) Patent Application Publication
(10) Pub. No.: US 2003/0166598 A1
(43) Pub. Date: Sep. 4, 2003

(54) Medicines for treating tumoral pathologies containing the ro5-4864 compound and an apoptosis-inducing agent

(76) Inventors: Didier Decaudin, Verrieres-Le-Buissen (FR); Guido Kroemer, Paris (FR); Marie-France Poupon, Fresnes (FR); Fariba Nemati, Paris (FR); Arnaud Beurdeley-Thomas, Paris (FR)

Correspondence Address: YOUNG & THOMPSON, 745 SOUTH 23RD STREET 2ND FLOOR, ARLINGTON, VA, 22202

(21) Appl. No.: 10/258,731

(22) PCT Filed: Apr. 27, 2001
(86) PCT No.: PCT/FR01/01322

(30) Foreign Application Priority Data
Apr. 28, 2000 (FR) 00/05438

(51) Int. Cl. A61K 48/00; A61K 31/5513

(52) U.S. Cl. 514/44; 514/221

(57) ABSTRACT

The invention concerns the use of Ro5-4864, and compounds derived therefrom, for preparing medicines for treating tumoral pathologies. The invention also concerns said compounds combined with an apoptosis-inducing agent, as combination products for simultaneous, separate or prolonged use, in cancer therapy.

The present invention relates to the use of the compound Ro5-4864, and of compounds derived from the latter, conforming to formula (I) below, for the preparation of medicines for the treatment of tumor pathologies.

At present, cancer therapy consists of the use of radiotherapy, chemotherapy, or a combination of these. Among the products used in chemotherapy are compounds that induce apoptosis, i.e. compounds that have the effect of stimulating programmed cell death. These methods have harmful side effects, and are poorly tolerated by patients.

The use of pharmacological agents that aim to increase the susceptibility of tumour cells to the induction of apoptosis would be very advantageous, as it would make it possible to substantially reduce the dose of compounds that are, directly or indirectly, inducers of apoptosis, used in radiotherapy or chemotherapy.

As such, the compound PK11195 of the following formula

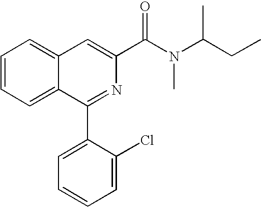

is described in international patent application WO 99/66958 for the preparation of medicines intended for the treatment of cancers, in combination with inducers of apoptosis.

The present invention follows from the demonstration by the Inventors, on an experimental animal model, that the compound Ro5-4864, and compounds derived from the latter conforming to formula (Ia) below, have an effect of stimulating apoptosis induced by apoptosis inducing compounds, greater than that observed with diazepam and the aforementioned compound PK11195.

The present invention relates to the use of at least one compound selected from those of the following formula (I)

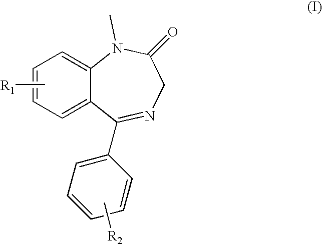

in which:

R₁ represents a hydrogen atom, or a halogen atom such as Cl,

R₂ represents a halogen atom such as Cl,

for the preparation of a medicine intended for the treatment of tumoral pathologies, the said compound preferably being combined with at least one apoptosis inducing agent, as combination products for simultaneous use, separate use or spread over time, in cancer therapy.

The invention relates more particularly to the aforementioned use of compound Ro5-4864 of the following formula:

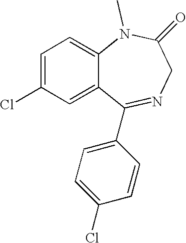

The invention relates more particularly to products comprising:

at least one compound of formula (I) above in which R₁ and R₂ represent a halogen atom such as Cl,

and at least one apoptosis inducing agent, as combination products for simultaneous use, separate use or spread over time, in cancer therapy.

Combination products as defined above that are preferred within the scope of the present invention are those containing, as compound of formula (I), the compound Ro5-4864, the formula of which is shown above.

The invention also relates to combination products as defined above, comprising at least one apoptosis inducing agent, if necessary inserted in a suitable vector for gene therapy, especially a vector of viral origin, the said agent being selected from those which damage the DNA, the natural or synthetic ligands of the receptor to glucocorticoids, or other pro-apoptotic compounds.

The invention relates more particularly to the combination products as defined above, characterized in that the apoptosis inducing agent is selected from:

derivatives of glucocorticoids, such as dexamethasone,

alkylating agents such as:

nitrogen mustards, especially cyclophosphamide,

platinum complexes,

derivatives of ethylene-imine,

derivatives of dimethane sulphonoxy-alkanes,

derivatives of piperazine,

inhibitors of topoisomerases such as:

inhibitors of topoisomerase 2, especially the anthracyclines, epipodophyllotoxin such as etoposide,

inhibitors of topoisomerase 1, especially camptothecine derivatives,

antimetabolites such as:

antifolates, especially methotrexate,

antipurines, especially 6-mercaptopurine,

antipyrimidines, especially 5-fluorouracil,

antimitotics such as:

vinca alkaloids,

taxoids, especially taxol, taxotere,

cytolytic compounds such as:

bleomycin,

dacarbazine,

hydroxycarbamide,

asparaginase,

mitoguazone,

plicamycin,

gamma rays,

etoposide,

doxorubicin,

compounds such as lonidamine or its derivatives,

monoclonal or other antibodies, or any compound or other treatment of immunotherapy.

Preferably, the aforementioned combination products of the invention contain a product of formula (I) in which R₁ and R₂ represent a halogen atom such as Cl, advantageously the compound RO5-4864, and an apoptosis inducing agent in a weight ratio from about 1:5 to about 1:1.

The preferred doses of RO5-4864 envisaged for human use are between 1 and 10 mg daily.

Advantageously, the aforementioned combination products of the invention also include one or more pharmaceutically acceptable vehicles, and are in a suitable form for oral or parenteral administration, especially by the intramuscular, intravenous or subcutaneous route.

The invention also relates to the use of the aforementioned combination products for the preparation of a medicine intended for the treatment of tumoral pathologies as defined above.

The invention will be further illustrated with the aid of the following description of the execution of tests for measuring the stimulating effect of the compound Ro5-4864 on apoptosis induced by apoptosis inducing compounds.

Material: female nude/nude mice, weight 30 g, age 6-8 weeks. Bred in conditions free from pathogens, with artificial lighting (12 hours of light, 12 hours of darkness).

Interscapular subcutaneous transplantation of the human tumours under investigation.

Treatment of the mice with an apoptosis inducing compound as defined above, and the compound Ro5-4864, when the tumours reach a diameter of about 5 mm (or a volume of 60 mm³).

Weekly weighing of the mice. Measurement of the tumours 3 times per week.

Analysis: measurement of tumour volume (V) and of relative tumour volume (RTV).

V=a²×b/2, where a is the largest diameter and b is the smallest diameter of the tumour.

RTV=Vx/Vi, where Vx is the mean volume at time x, and Vi is the mean volume at time D0.

Comparison of the results obtained for mice treated with the apoptosis inducing compound alone, with the results obtained on the one hand for mice treated with the apoptosis inducing compound and the compound Ro5-4864, and, on the other hand, for mice treated with the compound Ro5-4864 alone.

Sacrifice of the mice when the tumours reach a volume of 2500 mm³.

I. Introduction

Apoptosis, or programmed cell death, is controlled at the level of a common central effector located on the mitochondria (Kroemer and Reed, 2000). The mitochondrial membrane permeability (external membrane) constitutes the irreversible event that induces cell death. This permeability is controlled, on the one hand, by a multiprotein complex that permits opening or closing of membrane pores, and on the other hand by regulatory proteins such as Bcl-2 or Bax.

This multiprotein complex, called a permeability transition pore, contains the peripheral benzodiazepine receptor (PBR) (Zoratti and Szabo, 1995). There are two benzodiazepine binding sites: the central receptors localized in the CNS and on the cell plasma membrane, and the peripheral receptors localized principally on the external mitochondrial membrane. Furthermore, there are various types of PBR ligands: endogenous ligands (DBI or diazepam binding inhibitor and porphyrins) and exogenous ligands (benzodiazepines such as diazepam and 4′-chlorodiazepam (or RO5-4864) and isoquinoline carboxamides such as PK11195).

We demonstrated, for various cell lines, that the antitumoral effect of anti-Fas-receptor (FasR) antibodies was increased by PBR ligands (RO5-4864, diazepam and PK11195) and that this effect was far greater (by at least 3 or 4 times) with RO5-4864 than with the other compounds. In addition, only RO5-4864 is capable, on a line transfected by an apoptosis resistance gene such as the lines that over express the bcl-2 or bcl-X_{L} genes, of increasing the apoptotic effect of the anti-FasR antibodies. Furthermore, we demonstrated, on two human tumours of small-cell lung cancer xenografted into nude mice, that RO5-4864 increased the effect of antitumoral chemotherapy.

II. Material and Methods:

1. Cell Lines, Conditions of Culture and Induction of Apoptosis:

The human lines Jurkat (lymphoid T), SHEP (neuroblastoma) transfected with a control vector or with a bcl-2 or bcl-X_{L} vector, 143N2 (osteosarcoma) and SNB79 (glioblastoma) were cultured in DMEM or RPMI 1640 (Sigma Chemical Co., St Louis, Mo.) supplemented with FCS 10% (Dutscher, Brumath, France), penicillin G (10² IU/mI)+streptomycin (50 μg/ml) (Sigma) and L-glutamine (2 nM, Sigma).

The cells were cultured in the presence of anti-Fas receptor antibody CH11 (1 μg/ml; Immunotech, Marseilles, France) concomitantly or after exposure to RO5-4864 (BioBlock Scientific, Illkirch, France), diazepam (Roche, Neuilly sur Seine, France), or PK11195 (BioBlock), at various concentrations.

2. Quantification of Apoptosis and of Cell Viability:

Lipophilic fluorochrome DiOC₆(3) (Molecular Probes, Eugene, Oreg.) was used for measuring the membrane potential of the mitochondria (Δψₘ). Briefly, the cells were incubated for 15 min at 37° C. in the presence of 40 nM of DiOC₆(3), with immediate analysis of the incorporation of fluorescence on a type Epics Profile II cytofluorometer (Coulter, Miami, Fla.). Hydroethydine (HE) (2 μM, 15 min at 37° C., Molecular Probes) was used for measuring the production of superoxide anion (Marchetti et al., 1996). Finally, the proportion of cells that had lost part of the chromosomal DNA (subdiploid cells) was determined with propidium iodide on cells fixed in ethanol (Nicoletti et al., 1991).

Cell viability was measured by testing with methylene blue (Dimanche-Boitrel et al., 1992).

3. Studies in Vivo:

Female Swiss mice, nude/nude, age 6 to 8 weeks and weighing approx. 30 g, were used for the in vivo experiments. For the therapeutic tests, the mice grafted with human tumours were randomized in equivalent groups of 4 to 8 animals and were treated as soon as the tumour reached a diameter of 5 mm (i.e. an approximate volume of 60 mm³). Tumour growth was evaluated by measuring two tumour diameters at right angles.

Two human tumours of small-cell bronchial carcinoma were used: SCLC6 and SCLC61.

Chemotherapy, administered by intraperitoneal route, comprised either etoposide alone at a dose of 12 mg/kg/day D1 to D3, or a combination of etoposide 12 mg/kg/day+ifosfamide 90 mg/kg/day from D1 to D3. The RO5-4864 was prepared in an excipient containing ethanol+Tween 80 and was injected subcutaneously at a dose of 12 to 40 mg/kg/day from D1 to D3. The control group received injections of physiological serum.

4. Statistical Analyses:

A Student t-test was used for comparing the growth of the xenotransplanted tumours in the nude mice in the various randomized groups.

III. Results:

1. Studies in Vitro:

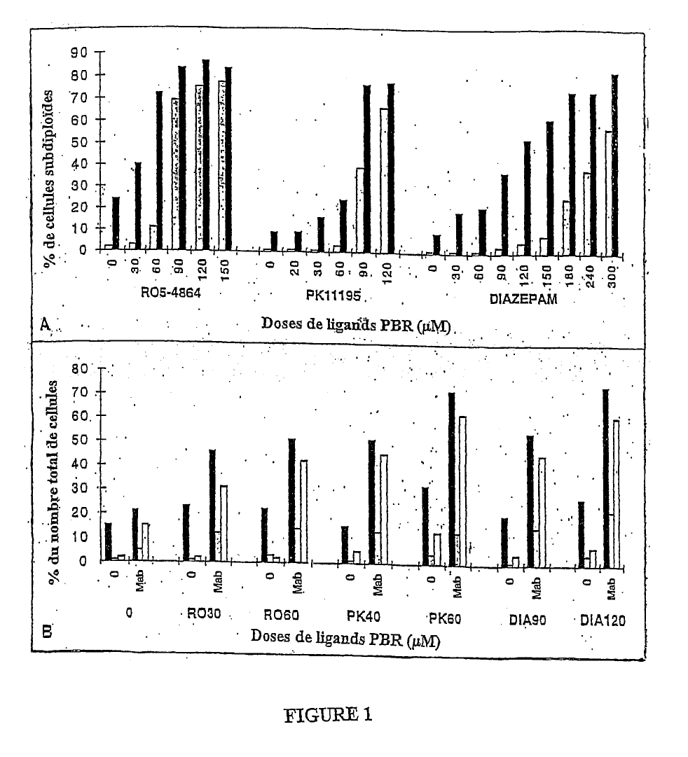

On the lymphoid line T Jurkat, RO5-4864, diazepam and PK11195 potentiate the apoptotic effect of the CH11 anti-FasR antibody (FIGS. 1A and 1B). This effect is greatest with RO5-4864 since, at an equivalent concentration of 60 μM, the proportion of subdiploid (apoptotic) cells is 73%, 25% and 20%. for RO5-4864, PK11195 and diazepam, respectively, in combination with CH11.

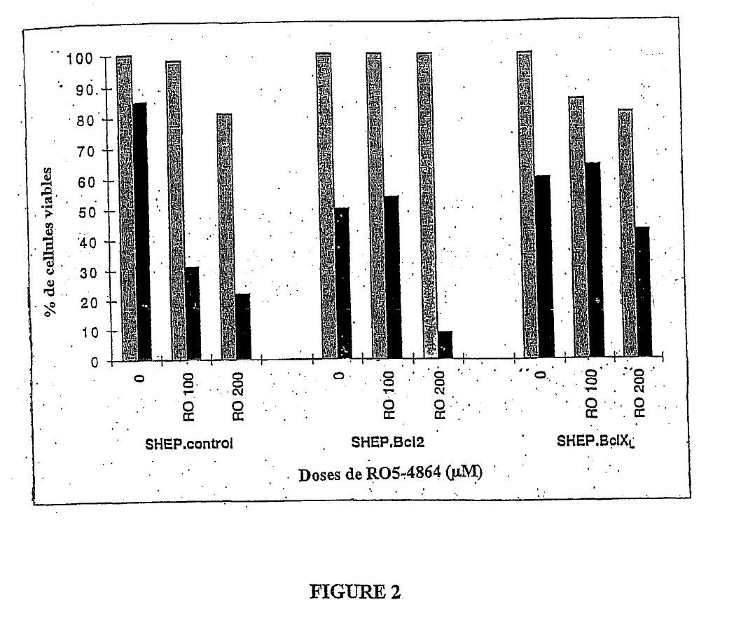

RO5-4864 reverses the resistance to the CH11 anti-FasR antibody of the lines SHEP-control, SHEP-bcl-2, or bcl-X_{L}, 143N2 and SNB79. On the other hand, this reversal is only observed with diazepam and PK11195 on the line SHEP-control (FIGS. 2, 3 and 4).

2. Studies in Vivo:

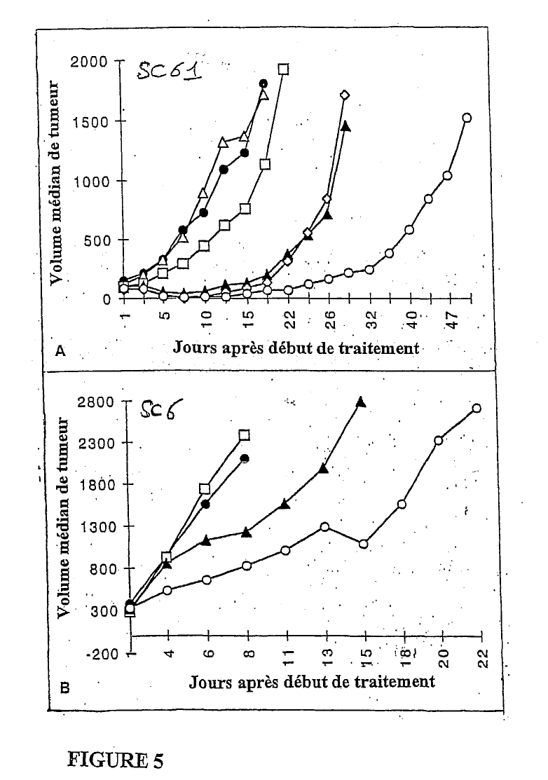

The mice with the transplanted human tumour SCLC61 were treated with etoposide injected by intraperitoneal route at a dose of 12 mg/kg/day from D1 to D3, with or without RO5-4864 at a dose of 12 mg/kg/day s.c. D1 to D3. Tumour growth was not altered by RO5-4864 alone. In contrast, the antitumour effect of etoposide is increased by concomitant administration of RO5-4864 (p<0.005) (FIG. 5A). The excipient of RO5-4864, injected alone or in combination with etoposide, does not alter the tumour growth of the control group and of the etoposide group.

The transplanted SCLC6 tumours were treated with the combination of etoposide 12 mg/kg/day+ifosfamide 90 mg/kg/day from D1 to D3, with or without RO5-4864 40 mg/kg/day from D1 to D3. The antitumour effect of the chemotherapy was increased by RO5-4864 (p<0.05) (FIG. 5B).

IV. Conclusion

Taken together, these experiments show that RO5-4864, on several types of human lines, is capable of raising resistance to anti-FasR antibodies and, on 2 tumours of small-cell lung cancer, of increasing the antitumour effect of chemotherapy combining etoposide±ifosfamide. Furthermore, on the SHEP human neuroblastoma line transfected with the bcl-2 or bcl-X_{L} genes, RO5-4864 reverses the resistance to anti-FasR antibodies.

Various experiments demonstrated that the effect of RO5-4864 was far greater than that of diazepam or of PK11195 and that, in particular, RO5-4864 was the only one capable of reversing the resistance to anti-FasR antibodies on the SHEP line transfected with the bcl-2 or bcl-X_{L} genes.

V. References:

Kroemer G, Reed J C. Mitochondrial control of cell death. Nat Med. 2000; 6: 513-19.

Zoratti M, Szabo I. The mitochondrial permeability transition. Biochem Biophys Acta Rev Biomembr 1995; 1241: 139-76.

Marchetti P, Hirsch T, Zamzami N, Castedo M, Decaudin D, Susin S A, et al. Mitochondrial permeability transition triggers lymphocyte apoptosis. J Immunol 1996; 157: 4830-6.

Nicoletti I, Migliorati G, Plagliacci M C, Riccardi C. A rapid simple method for measuring thymocyte apoptosis by propidium iodide staining and flow cytometry. J Immunol Methods 1991; 139: 271-80.

Dimanche-Boitrel M-T, Pelletier H, Genne P, Petit J-M, Le Grimellec C, Canal P, et al. Confluence-dependent resistance in human colon cancer cells: role of reduced drug accumulation and low intrinsic chemosensitivity of resting cells. Int. J. Cancer 1992; 50: 677-82.

### VI. FIGURE CAPTIONS

1. FIG. 1: culture of Jurkat cells in the presence of CH11 with (white histograms) or without (black histograms) RO5-4864, PK11195 or diazepam. In A, determination of the proportion of subdiploid cells (ordinate), as a function of the concentration in μM of the RBP ligands RO5-4864, PK11195 and DIAZEPAM. In B, determination of the percentage of cells (ordinate), with decrease of the (Δψm) (black histograms), production of superoxide anion (grey histograms) and subdiploids (white histograms), in the absence (o) or in the presence of antiFas CH11 antibodies (still designated Mab); as a function of the concentration in μM of the RBP ligands RO5-4864 (at 30 μM or RO30, and at 60 μM or RO60), PK11195 (at 40 μM or PK40, and at 60 μM or PK60) and DIAZEPAM (at 90 μM or DIA90, and at 120 μM or DIA120).

2. FIG. 2: culture of the SHEP control lines (SHEP.control), of SHEP lines transfected with bcl2 (SHEP.Bcl2), and of SHEP lines transfected with bclX_{L} (SHEP.BClX_{L}), in the presence of CH11 with (black histograms) or without (grey histograms) RO5-4864. Evaluation of cell viability (test with methylene blue; percentage of cells on ordinate), as a function of the concentration of RO5-4864 (0, 100 μM or RO100, 200 μM or RO 200).

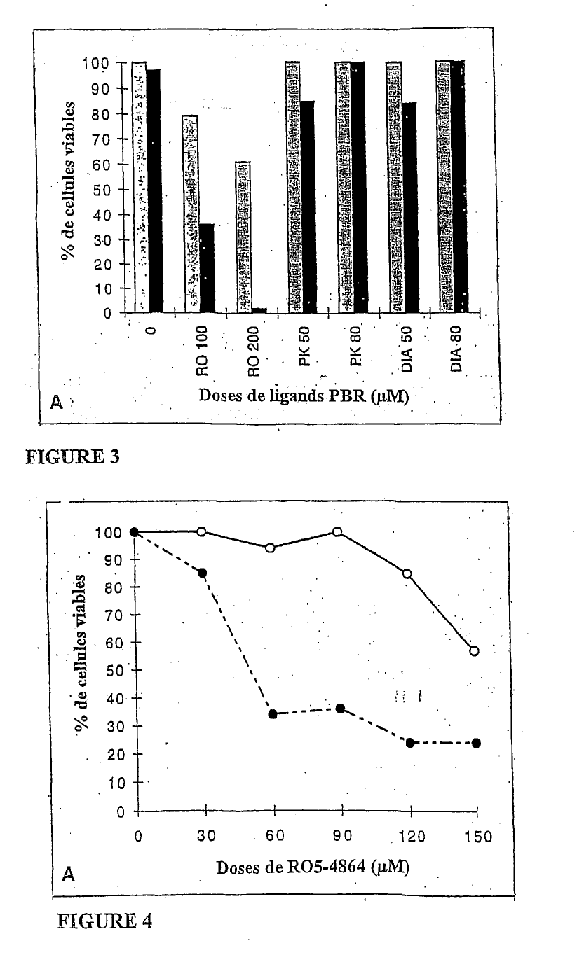

3. FIG. 3: culture of the 143N2 line in the presence of CH11 with (black histograms) or without (grey histograms) RO5-4864, PK11195 or diazepam. Evaluation of cell viability (test with methylene blue; percentage of cells on ordinate), as a function of the concentration of RO5-4864 (100 μM or RO 100, 200 μM or RO 200), PK11195 (50 μM or PK50, 80 μM or PK 80) and DIAZEPAM (50 μM or DIA50, 80 μM or DIA80).

4. FIG. 4: culture of the SNB79 line in the presence of CH11 with (•) or without (o) RO5-4864. Evaluation of cell viability (test with methylene blue; percentage of cells on the ordinate), as a function of the concentration (μM) of Ro5-4864.

5. FIG. 5: in A, SCLC61 tumour xenotransplanted in the nude mouse and treated with etoposide with (o) or without ( ) RO5-4864. Two control groups received the excipient of RO5-4864 alone (Δ) or with etoposide (⋄). In B, SCLC6 tumour xenotransplanted in the nude mouse and treated with etoposide+ifosfamide with (o) or without ( ) RO5-4864. The mean volume of the tumours is shown on the ordinate as a function of the number of days from the start of treatment. Two control groups received injections either of RO5-4864 alone (□), or of NaCl 0.9% (•).

1. Products comprising:
at least one compound of the following formula (I) 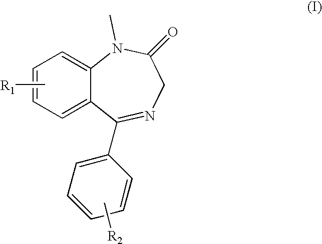
in which R₁ and R₂ represent a halogen atom such as Cl,
and at least one apoptosis inducing agent, as combination products for simultaneous use, separated or spread out over time, in cancer therapy.

2. A product according to claim 1, characterized in that it contains, as compound of formula (I), the compound Ro5-4864 of the following formula:

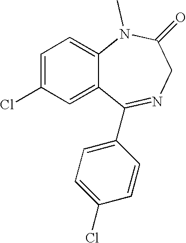

3. A product according to claim 1 or 2, in a form that can be administered orally or parenterally, especially by intramuscular or intravenous route.

4. A product according to one of claims 1 to 3, characterized in that it contains at least one apoptosis inducing agent, if necessary inserted in a suitable vector for gene therapy, especially a vector of viral origin, the said agent being selected from those that damage the DNA, the natural or synthetic ligands of the receptor to glucocorticoids, or other pro-apoptotic compounds.

5. A product according to one of claims 1 to 4, characterized in that the apoptosis inducing agent is selected from:
derivatives of glucocorticoids, such as dexamethasone,
alkylating agents such as:
nitrogen mustards, especially cyclophosphamide,
platinum complexes,
derivatives of ethylene-imine,
derivatives of dimethane sulphonoxy-alkanes,
derivatives of piperazine,
inhibitors of topoisomerases such as:
inhibitors of topoisomerase 2, especially the anthracyclines, epipodophyllotoxin such as etoposide,
inhibitors of topoisomerase 1, especially camptothecine derivatives,
antimetabolites such as:
antifolates, especially methotrexate,
antipurines, especially 6-mercaptopurine,
antipyrimidines, especially 5-fluorouracil,
antimitotics such as:
vinca alkaloids,
taxoids, especially taxol, taxotere,
cytolytic compounds such as:
bleomycin,
dacarbazine,
hydroxycarbamide,
asparaginase,
mitoguazone,
plicamycin,
compounds such as lonidamine or derivatives,
or monoclonal or other antibodies, or any compound or other treatment of immunotherapy.

6. A product according to one of claims 1 to 5, characterized in that it contains at least one apoptosis inducing agent selected from:
gamma rays,
etoposide,
doxorubicin,
dexamethasone,
lonidamine.

7. Products according to one of claims 1 to 6, characterized in that they contain a product of formula (I) and an apoptosis inducing agent in a weight ratio from about 1:5 to about 1:1.

8. Products according to one of claims 1 to 7, characterized in that they also comprise one or more pharmaceutically acceptable vehicles.

9. Use of products according to one of claims 1 to 8, for the preparation of a medicine for treating tumoral pathologies.
% of subdiploid cells
Concentration of RBP ligands (μM)
% of cells
Concentration of RBP ligands (μM)
% of viable cells
Concentration of RO5-4864 (μM)
% of viable cells
Concentration of RBP ligands (μM)
% of viable cells
Concentration of RO5-4864 (μM)
Mean volume of tumor
Mean volume of tumor
Number of days from the start of treatment
Number of days from the start of treatment
